(19) United States
(12) Patent Application Publication
(10) Pub. No.: US 2026/0272833 A1
(43) Pub. Date: Sep. 17, 2026

(54) ORAL DRUG-LOADED MICELLE COMPOSITION AND PREPARATION METHOD THEREOF

(71) Applicant: Shanghai Ninth People's Hospital, Shanghai Jiaotong University School of Medicine, Shanghai (CN)

(72) Inventors: Tianhai JI, Shanghai (CN); Wenqing ZANG, Shanghai (CN)

(21) Appl. No.: 19/124,607

(22) PCT Filed: Jan. 23, 2024
(86) PCT No.: PCT/CN2024/073715

§ 371 Date: Apr. 25, 2025

(30) Foreign Application Priority Data
Nov. 23, 2022 (CN) 202211476418.0

(51) Int. Cl. A61K 9/107; A61K 9/00; A61K 31/7068; A61K 47/34

(52) U.S. Cl. CPC A61K 9/1075; A61K 9/0053; A61K 31/7068; A61K 47/34

(57) ABSTRACT

An oral drug-loaded micelle composition and a preparation method thereof. The oral drug-loaded micelle composition includes a PLGA-PEG high polymer modified by glycocholic acid, marked as PLGA-PEG-GCA; and a hydrophobic small molecule drug, wherein a hydrophobic end PLGA of GCA-PLGA-PEG is aggregated to wrap the hydrophobic small molecule drug, and a hydrophilic end of the GCA-PLGA-PEG extends toward a direction away from the hydrophobic end PLGA to form a spherical drug-loaded micelle. Accordingly, the PLGA-PEG high polymer modified by glycocholic acid is adopted to wrap the hydrophobic small molecule drug, such that a micelle-based nano-drug is obtained. Experiments prove that the drug-loaded micelle composition disclosed by the present invention improves the oral bioavailability of the hydrophobic small molecule drug, and can achieve a better anti-tumor treatment effect while reducing an administration dose.

### TECHNICAL FIELD

The present invention belongs to the field of pharmaceutical preparations, and more particularly relates to an oral drug-loaded micelle composition and a preparation method thereof.

### BACKGROUND ART

Since the discovery in the 1940s that nitrogen mustard could be used for the treatment of malignant tumors, chemotherapy drugs have made considerable progress in recent decades. Antimetabolites play an important role in the field of cancer and other tumor chemotherapy and are increasingly approved by the FDA. The antimetabolites have similar chemical structures to metabolites, and can competitively bind to metabolically essential enzymes to inhibit metabolic pathways of purines, pyrimidines and pyrimidine nucleosides, etc.; or as pseudo-metabolites to form false non-functional biological macromolecules with DNA, that is, to lead to so-called lethal synthesis, so that tumor cells lose their functions and die. Gemcitabine has the following structural formula:

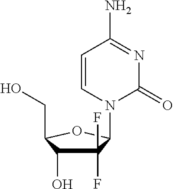

Gemcitabine with CAS number: 95058-81-4, as a cytosine derivative antimetabolite drug, has been approved by the FDA since 1996, and has been widely applied in the treatment of a variety of diseases, including cancers, due to its characteristics of low price and easy availability. This drug can be converted into an active nucleoside triphosphate analogue within cells to inhibit DNA polymerase and block DNA synthesis, thereby inhibiting the growth of tumor cells. However, during actual use, the efficacy of this drug will be greatly compromised by various factors. One typical drawback is that this drug cannot be taken orally. Gemcitabine and its analogues are prone to undergo first-pass metabolism in the liver by deamination with cytosine deaminase due to the presence of a No. 4 amino group of a cytosine fragment, and are converted into inactive uracil gemcitabine, resulting in poor oral efficacy of gemcitabine. Therefore, gemcitabine is usually administered by intravenous continuous infusion. However, this administration mode has a significant impact on convenience and clinical applications.

To overcome the aforementioned problems, researchers have conducted extensive studies and modifications on its structure in an attempt to achieve oral administration, most of which focus on prodrug strategies. However, the direct chemical structural modification of gemcitabine often requires more complex synthetic routes; and due to the structural changes in active sites of small molecules, may lead to unexpected side effects. In 2009, Eli Lilly and Company modified a cytosine amino group into valpromide to obtain a prodrug LY2334737, which improved the oral bioavailability of gemcitabine, such that relatively stable amide bonds could be slowly hydrolyzed under the action of carboxylesterase in vivo to exert its efficacy. Such structural modification effectively attenuates the first-pass metabolic effect of hepatic deaminase on gemcitabine-based drugs. However, due to serious toxic and side effects of this candidate compound in clinical trials, Eli Lilly and Company terminated its research and development work in 2013. As of today, the clinical issues regarding the oral administration of gemcitabine-based drugs remain unsolved effectively.

### SUMMARY OF THE INVENTION

An object of the present invention is to solve the problem that gemcitabine-based drugs are difficult to be administered orally, and to provide a drug-loaded micelle composition, in which a drug delivery carrier is modified by glycocholic acid (GCA), and an intestinal bile acid transporter is used to improve the oral availability of the drugs, thereby achieving oral administration.

To fulfill said object, the present invention provides an oral drug-loaded micelle composition, which includes:
a PLGA-PEG high polymer modified by glycocholic acid (GCA), marked as PLGA-PEG-GCA; and
a hydrophobic small molecule drug, wherein
a hydrophobic end PLGA of GCA-PLGA-PEG is aggregated to wrap the hydrophobic small molecule drug, and a hydrophilic end of the GCA-PLGA-PEG extends toward a direction away from the hydrophobic end PLGA to form a spherical drug-loaded micelle.

Optionally, in PLGA-PEG-GCA, a PLGA fragment has a molecular weight of 8000 Da-12000 Da; and in the PLGA-PEG-GCA, a PEG fragment has a molecular weight of 3000 Da-7000 Da.

Optionally, the spherical drug-loaded micelle has a particle size of 20-200 nm.

Optionally, the hydrophobic small molecule drug includes one or more of the followings: gemcitabine, doxorubicin, daunorubicin, epirubicin, azathramycin, valrubicin, anthracyclines, actinomycin-D, bleomycin, mitomycin-C, cyclophosphamide, mecloxamine, uramustine, melphalan, chloramphenicol, ifosfamide, bendamustine, carmustine, lomustine, streptomycin, busulfan, dacarbazine, temozolomide, thio-TEPA, altretamine, cisplatin, carboplatin, nedaplatin, oxaliplatin, satraplatin, triplatinum tetranitrate, 5-fluorouracil, 6-mercaptopurine, capecitabine, cladribine, clofarabine, cytarabine, fluorouracil, fludarabine, hydroxyurea, methotrexate, pemetrexed, pentostatin, thioguanine; camptothecin, topotecan, irinotecan, etoposide, teniposide, mitoxantrone, paclitaxel, docetaxel, ixabepilone, vinblastine, vincristine, vindesine, vinorelbine, estradiol and derivatives thereof.

Optionally, the hydrophobic small molecule drug in the oral drug-loaded micelle has a drug loading rate of 11%-20% by mass.

Optionally, the hydrophobic small molecule drug in the oral drug-loaded micelle has an encapsulation rate of 70%-90% by mass.

The present invention further provides a preparation method of the oral drug-loaded micelle composition. The preparation method includes:
step 1, preparing PLGA-PEG-GCA from PLGA-PEG-COOH modified by glycocholic acid (GCA); and
step 2, adding a hydrophobic small molecule drug and PLGA-PEG-GCA to an organic solvent in a mass ratio of 1:5 to 1:10, which are dissolved, then dropwise added to distilled water under the action of ultrasounds, emulsified to form a micelle for wrapping the hydrophobic small molecule drug, purified and filtered to obtain the oral drug-loaded micelle.

Optionally, the step 1 may include:
step 1.1, with glycocholic acid (GCA) and ethylenediamine (EDA) as raw materials, reacting at room temperature under the action of a condensing agent to prepare GCA-EDA; and
step 1.2, activating a carboxyl group of PLGA-PEG-COOH in the atmosphere of nitrogen, adding GCA-EDA, reacting at room temperature for 6 h-12 h, and purifying to obtain the PLGA-PEG-GCA.

Optionally, the purifying step includes: performing dialysis by distilled water at a molecular weight of 12 k Da-16 k Da and purifying for 24 h-72 h to remove the hydrophobic small molecule drug free outside the micelle.

Optionally, PLGA is also added in the step 2.

According to the present invention, the PLGA-PEG high polymer modified by glycocholic acid is adopted to wrap the hydrophobic small molecule drug (e.g., gemcitabine), such that a micelle-based nano-drug is obtained. Through pharmacokinetic experiments in rats, it has been proved that it can improve the oral bioavailability of gemcitabine. Through in vitro cytotoxicity tests and in vivo pharmacodynamic experiments in mice, it has been proved that the oral administration of a chemotherapy drug gemcitabine can be realized and a therapeutic effect on pancreatic cancers can be exerted, while an administration dose is reduced.

### BRIEF DESCRIPTION OF THE DRAWINGS

FIG. 1 is a schematic structural diagram of an oral drug-loaded micelle composition of the present invention.

FIG. 2 is a 1H NMR spectrum of a PLGA_{10K}-PEG₅ₖ-GCA (PPG) polymer of the present invention.

FIG. 3 is TEM electron microscope diagrams of three types of gemcitabine micelles prepared in Examples 1-2 and Comparative example 1 of the present invention. A represents Gem-PPG60, B represents Gem-PPG100, and C represents Gem-PP100.

FIG. 4 is in vitro release test diagrams of three types of gemcitabine micelles prepared in Examples 1-2 and Comparative example 1 of the present invention.

FIG. 5 is a schematic diagram of the progression of tumor sizes of BxPC-3 tumor-bearing mice within 33 days.

FIG. 6 is a schematic diagram of weight changes of BxPC-3 tumor-bearing mice within 33 days.

### DETAILED DESCRIPTION OF THE INVENTION

The enterohepatic circulation of bile acid consists of two main processes: hepatic secretion and intestinal absorption. The bile acid is secreted into the duodenum and emulsifies water-insoluble nutrients to facilitate intestinal absorption. In the distal small intestine, the bile acid is absorbed by passive diffusion and active transport. Passive diffusion occurs in proximal regions of the small intestine and colon, while active transport is only restricted to the ileum. In human beings and other vertebrates, the ileal epithelium has evolved an effective transport mechanism to recycle the bile acid.

In order to solve the problem of poor oral effect of gemcitabine, the present invention develops an oral drug preparation based on an intestinal bile acid transport mechanism, and develops a drug-loaded nanoparticle modified by a bile acid derivative glycocholic acid, which interacts with a bile acid transport protein ASBT on a small intestinal epithelial membrane to undergo active transport, thereby promoting the uptake, transport and absorption of a loaded small molecule drug.

Glycocholic acid (GCA), as an important derivative of the bile acid, is the most abundant component in human bile, which has a relatively low log P value and can be more exposed to an aqueous phase. As a result, GCA can be more effectively taken up and transported by the bile acid transport protein ASBT on the intestinal membrane. Therefore, according to the present invention, a GCA-modified nanoparticle is used to load gemcitabine, which can greatly promote the active transport and absorption of a micelle loaded with a small molecule drug gemcitabine, thereby significantly improving the oral bioavailability of gemcitabine. In order to enhance a sustained-release effect of drug molecules, for a drug-loaded nanoparticle that requires GCA modification, a biodegradable medical high molecular polymer may be used as a drug delivery carrier. The present invention is exemplified by taking a PLGA-PEG high polymer as a drug molecule carrier.

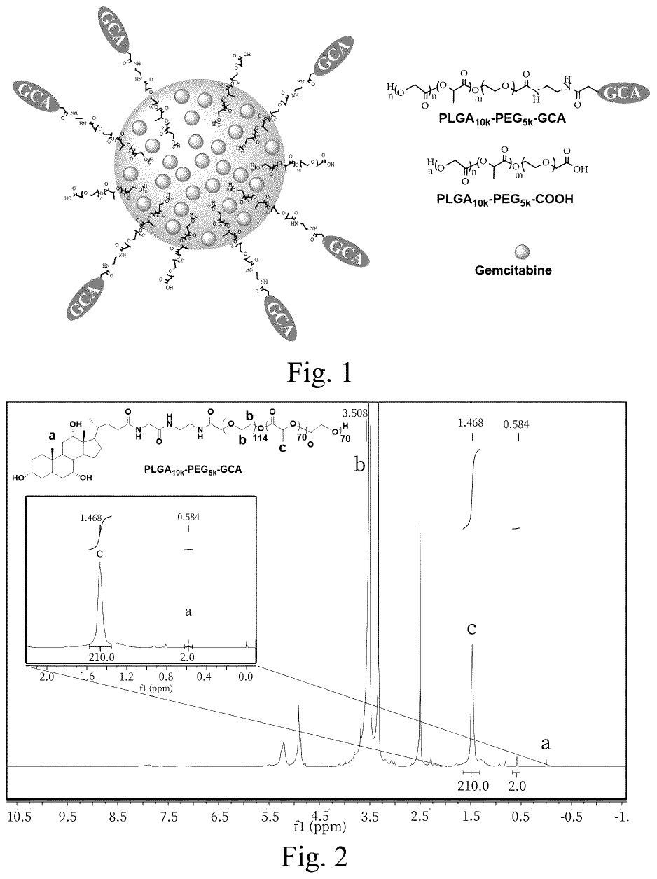

FIG. 1 shows a schematic structural diagram of an oral gemcitabine PPG micelle of the present invention. A PLGA-PEG polymer monomer PLGA-PEG-GCA surface-modified by glycocholic acid (GCA) (PPG for short) contains a hydrophilic end GCA and a hydrophobic end PLGA. Based on the principle of “like dissolves like”, the hydrophobic end aggregates into a micelle due to a hydrophobic-hydrophobic interaction, while the hydrophilic end GCA extends toward a direction away from the hydrophobic end PLGA due to its hydrophilicity and large steric hindrance. According to the same principle, the hydrophobic small molecule gemcitabine is prone to accumulation at the hydrophobic end PLGA, that is, being loaded into PPG to form a spherical drug-loaded micelle composition.

It may be understood that, in addition to gemcitabine, other hydrophobic small molecule drugs, e.g., gemcitabine, doxorubicin, daunorubicin, epirubicin, azathramycin, valrubicin, anthracyclines, actinomycin-D, bleomycin, mitomycin-C, cyclophosphamide, mecloxamine, uramustine, melphalan, chloramphenicol, ifosfamide, bendamustine, carmustine, lomustine, streptomycin, busulfan, dacarbazine, temozolomide, thio-TEPA, altretamine, cisplatin, carboplatin, nedaplatin, oxaliplatin, satraplatin, triplatinum tetranitrate, 5-fluorouracil, 6-mercaptopurine, capecitabine, cladribine, clofarabine, cytarabine, fluorouracil, fludarabine, hydroxyurea, methotrexate, pemetrexed, pentostatin, thioguanine; camptothecin, topotecan, irinotecan, etoposide, teniposide, mitoxantrone, paclitaxel, docetaxel, ixabepilone, vinblastine, vincristine, vindesine, vinorelbine, estradiol and derivatives thereof, etc., can also form oral drug-loaded PPG micelles through the above hydrophobic-hydrophobic interaction. The drug-loaded PPG micelle, based on surface modification with glycocholic acid (GCA), can also greatly promote active transport and absorption of the loaded small molecule drug in the small intestine, thereby significantly improving the oral bioavailability of this drug. In order to control the particle size of the micelle to be mainly distributed in the range of 20-200 nm, in PLGA-PEG-GCA, a PLGA fragment may have a molecular weight of 8000 Da-12000 Da, in this case 10000 Da, denoted as PLGA₁₀ₖ; and a PEG fragment may have a molecular weight of 3000 Da-7000 Da, in this case 5000 Da, denoted as PEG₅ₖ. In the oral drug-loaded micelle, the hydrophobic small molecule drug has a drug loading rate of 11%-20% by mass; and the hydrophobic small molecule drug has an encapsulation rate of 70%-90% by mass.

The present invention further provides a preparation method of the above oral drug-loaded micelle composition. The preparation method includes:
Step 1, preparing PLGA-PEG-GCA (PPG) from PLGA-PEG-COOH modified by glycocholic acid (GCA). GCA may be modified to PLGA-PEG-COOH in the form of an amide by means of conventional methods. In this case, the formation of the amide is promoted by adding a condensing agent. Specifically, step 1 includes:
Step 1.1, with glycocholic acid (GCA) and ethylenediamine (EDA) as raw materials, reacting at room temperature under the action of the condensing agent to prepare GCA-EDA. In this case, dicyclohexylcarbodiimide (DCC) is selected as the condensing agent. It may be understood that other conventional condensing agents may also be available, and the examples of the present invention are merely examples and are not intended to be restrictive. In this step, EDA may be excessive, for example, GCA: EDA may be 1:5 to 1:50 by mass.
Step 1.2, activating a carboxyl group of PLGA-PEG-COOH in the atmosphere of nitrogen, adding GCA-EDA, reacting at room temperature for 6 h-12 h, and purifying to obtain the PLGA-PEG-GCA. The carboxyl activation may be done by means of conventional activation methods. In this case, a carboxyl group of PLGA-PEG-COOH is activated by using 1-(3-dimethylaminopropyl)-3-ethylcarbodiimide hydrochloride (EDC) and N-hydroxysuccinimide (NHS) as activators, and then reacted with GCA-EDA at room temperature to form surface-modified PLGA-PEG-GCA.
Step 2, adding a hydrophobic small molecule drug and PLGA-PEG-GCA to an organic solvent in a mass ratio of 1:5 to 1:10, which are dissolved, then dropwise added to distilled water (the amount of distilled water is insignificant, as long as the purpose of emulsification can be achieved, and in this case, a mass ratio of the hydrophobic small molecule drug to the distilled water is 1:10000) under the action of ultrasounds, emulsified to form a micelle for wrapping the hydrophobic small molecule drug, purified and filtered to remove macromolecular aggregates of the hydrophobic small molecule drug and/or PLGA-PEG-GCA free outside the micelle, thereby obtaining the oral drug-loaded micelle.

The purifying step includes: performing dialysis by distilled water at a molecular weight of 12 k Da-16 k Da and purifying for 24 h-72 h to remove the hydrophobic small molecule drug free outside the micelle. In order to the particle size of the micelle, in step 2, PLGA is also added, and a molecular weight of PLGA can be selected according to the particle size, e.g., 8000 Da-12000 Da, in this case, 10000 Da. PLGA is hydrophobic. Based on the hydrophobic-hydrophobic interaction, after PLGA is added to a mixed solution of the hydrophobic small molecule drug and the PLGA-PEG-GCA, the hydrophobic-hydrophobic interaction between a high polymer and the small molecule drug is further enhanced, so that the high polymer and the small molecule drug are combined more closely, thereby reducing voids in the formed micelle and reducing the particle size of the micelle. The smaller the particle size of the micelle, the stronger the adsorption with cells, and the greater the probability of micelle endocytosis, such that the micelle has better oral bioavailability and pharmacokinetic properties.

The technical solutions of the present invention will be described clearly and completely in conjunction with the drawings. Obviously, the described examples are merely some examples, rather than all examples, of the present invention. Based on the examples of the present invention, all other examples derived by a person of ordinary skill in the art without creative efforts shall fall within the protection scope of the present invention.

### I. Preparation Methods of Micelle and its Polymerization Unit

1. Material sources

Glycocholic acid (GCA), dicyclohexylcarbodiimide (DCC), ethylenediamine (EDA), N-hydroxysuccinimide (NHS), ethyl acetate, dimethylformamide (DMF) and methanol were purchased from Aladdin® (Shanghai, China). 1-(3-Dimethylaminopropyl)-3-ethylcarbodiimide hydrochloride (EDC), dimethyl sulfoxide (DMSO) and DMSO-d₆ were purchased from Bidepharm® (Shanghai, China). PLGA₁₀ₖ and PLGA_{10K}-PEG₅ₖ-COOH were purchased from Tanch Tech® (Guangzhou, China), gemcitabine was purchased from Selleck Chemicals (Shanghai, China), and a gemcitabine hydrochloride solution was purchased from CTTQ Pharma (Nanjing, China).
2. Two-step synthesis method of PLGA-PEG polymer (PPG) modified by glycocholic acid (GCA) A synthesis route of PPG was as follows.

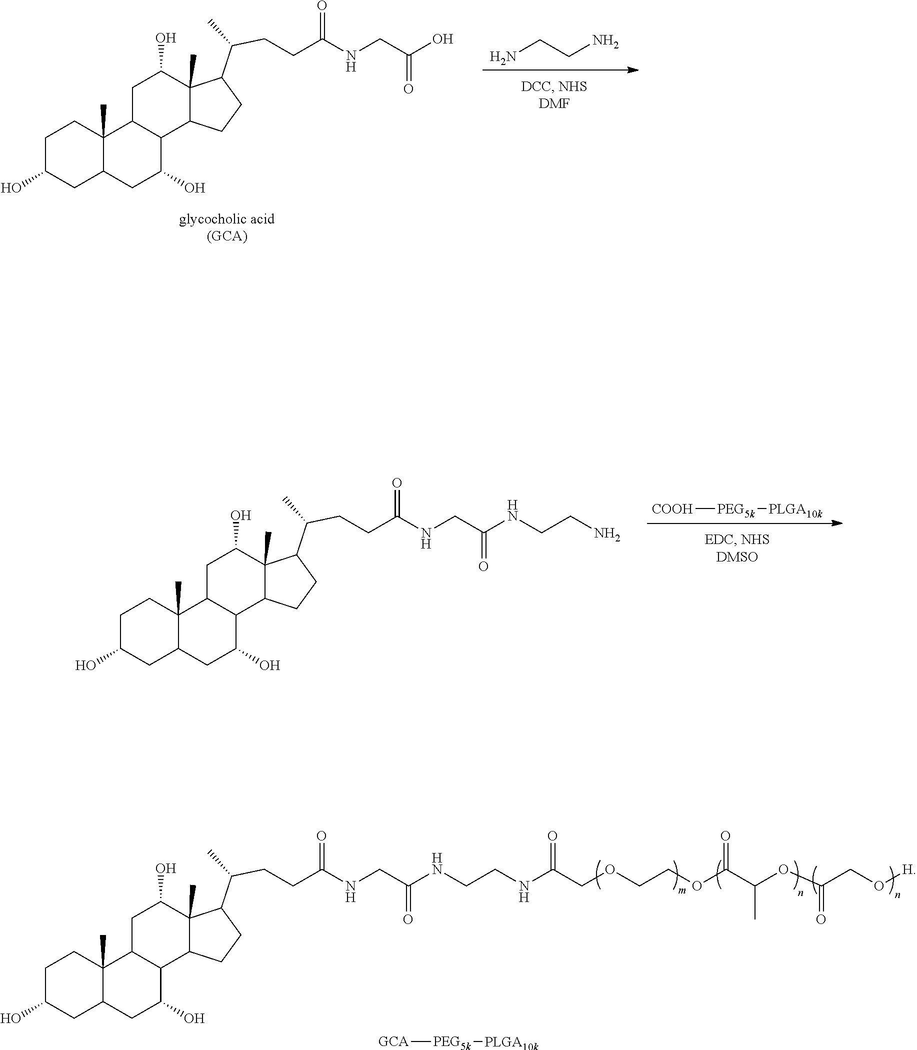

Step 1, GCA (500 mg, 1.0 equivalent), DCC (160 mg, 1.3 equivalent), and EDA (3.2 g, 50 equivalent) were dissolved in 10 mL of dry DMF and reacted at 35° C. for 24 h while stirring. The reaction solution was filtered and unreacted EDA was removed in vacuum. Then, the filtrate was precipitated in ethyl acetate and filtered, and particles were collected, then washed with EA and dried in vacuum for 24 h to obtain a dry GCA-EDA powder.
Step 2, PLGA_{10K}-PEG₅ₖ-COOH (600 mg, 1.0 equivalent), EDC (15.3 mg, 2.0 equivalent), and NHS (9.2 mg, 2.0 equivalent) were added to 10 mL of dimethyl sulfoxide in the atmosphere of nitrogen. The mixture was stirred at 30° C. for 4 h to activate a carboxyl moiety, and then added with GCA-EDA (60.8 mg, 3.0 equivalent) prepared in step 1 along with 10 μL of distilled EDA; and the reaction mixture was stirred again for 10 h. The reaction solution was purified by methanol dialysis (molecular weight cut-off of 1000 Da), dried in vacuum, redissolved in 10 mL of distilled water, lyophilized, and stored at −20° C. to obtain a PLGA-PEG polymer PPG modified by GCA.
3. Preparation of three types of gemcitabine-loaded PLGA-PEG micelles (Gem-PPG60, Gem-PPG100 and Gem-PP100)

Gem-PPG60, Gem-PPG100 and Gem-PP100 micelles were prepared by means of an ultrasonic method, respectively.

Example 1: preparation of gemcitabine-loaded PPG micelle (Gem-PPG60) having particle size of 60 nm Gemcitabine (6 mg), PLGA₁₀ₖ (6 mg) and synthesized PPG (30 mg) were dissolved in 1 mL of dimethyl sulfoxide. Under the action of ultrasounds, the mixture was added dropwise to 20 mL of distilled water within 5 minutes and emulsified to form a gemcitabine-trapped micelle Gem-PPG. Then, the obtained Gem-PPG micelle was dialyzed with distilled water (molecular weight of 14 k Da) and purified for 24 h to remove gemcitabine free outside the micelle. The purified Gem-PPG micelle solution was filtered through a 450 nm filter (Whatman Nucleopore, UK) and the filtrate was collected. The filtrate was ultrafiltered at a speed of 1500 r/min (Millipore, USA) to remove PPG that did not form the micelle, and the filtrate was removed. The ultrafiltered mixture from which the filtrate was removed was filtered again through a 220 nm filter to remove micelle aggregates, and the filtrate was a purified Gem-PPG60 micelle.

The purified Gem-PPG60 micelle was stored at 4° C.

Example 2: preparation of gemcitabine-loaded PPG micelle (Gem-PPG100) having particle size of 100 nm Gemcitabine (3 mg) and synthesized PPG (30 mg) were dissolved in 1 mL of DMSO. Under the action of ultrasounds, the mixture was added dropwise to 20 mL of distilled water within 5 minutes and emulsified to form a gemcitabine-trapped micelle Gem-PPG. Then, the obtained Gem-PPG micelle was dialyzed with distilled water (molecular weight of 14 k Da) and purified for 24 h to remove free gemcitabine. The purified Gem-PPG micelle solution was filtered through a 450 nm filter (Whatman Nucleopore, UK) and the filtrate was collected. The filtrate was ultrafiltered at a speed of 1500 r/min (Millipore, USA) to remove PPG that did not form the micelle, and the filtrate was removed. The ultrafiltered mixture from which the filtrate was removed was filtered again through a 220 nm filter to remove micelle aggregates, and the filtrate was a purified Gem-PPG100 micelle.

The purified Gem-PPG100 micelle was stored at 4° C.

Comparative example 1: preparation of gemcitabine-loaded PP micelle (Gem-PP100) having particle size of 100 nm

Gemcitabine (3 mg) and PLGA₁₀ₖ-PEG₅ₖ-COOH (30 mg) were dissolved in 1 mL of DMSO. Under the action of ultrasounds, the mixture was added dropwise to 20 mL of distilled water within 5 minutes and emulsified to form a gemcitabine-trapped micelle Gem-PP. Then, the obtained Gem-PP micelle was dialyzed with distilled water (molecular weight cutoff of 14 k Da membrane) and purified for 24 h to remove free gemcitabine. The purified crude Gem-PPG micelle solution was filtered through a 450 nm filter (Whatman Nucleopore, UK) and the filtrate was collected. The filtrate was ultrafiltered at a speed of 1500 r/min (Millipore, USA) to remove PPG that did not form the micelle, and the filtrate was removed. The ultrafiltered mixture from which the filtrate was removed was filtered again through a 220 nm filter to remove micelle aggregates, and the filtrate was a purified Gem-PP100 micelle.

The purified Gem-PP100 micelle was stored at 4° C.

The Gem-PPG60 micelle prepared in Example 1, the Gem-PPG100 micelle prepared in Example 2 and the Gem-PP100 micelle prepared in Comparative example 1 were physically characterized and tested for efficacy, respectively.

### II. Characterization of Physical Properties of Micelles

### 1. Grafting Rate Determination for PLGA-PEG Polymer (PPG) Unit Modified by GCA

10 mg of GCA-PEG₅ₖ-PLGA1ok was dissolved in 0.45 mL of deuterated DMSO-d₆ and measured on a Varian Unity 400 MHz NMR spectrometer. As shown in FIG. 2, three characteristic peaks a(GCA), b(PEG) and c(PLGA) of a PPG high polymer were δ 0.584 (a, CH₃, 2H), 1.468 (c, CH₃, 210H) and 3.508 (b, CH₂, 456H), respectively. Based on the known molecular weights of PEG (5 k Da) and PLGA (10 k Da), a GCA grafting rate was calculated as 2.0/(210/70)=66.7% based on ¹H NMR integration.

### 2. Morphological Characterization of Micelles

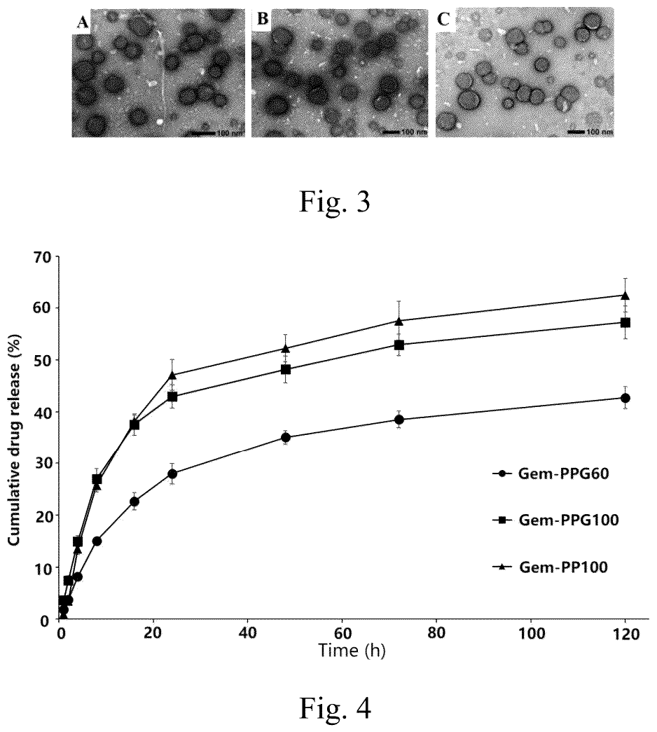

The morphologies of the micelles prepared in Examples 1-2 and Comparative example 1 were observed by a transmission electron microscope at an excitation voltage of 120 kV. A preparation method of a sample was as follows: 10 μL of micelle sample with a concentration of about 1 mg/mL was dropped on a TEM copper grid, placed in a drier for 8 h and dried naturally, and then stained with 1% uranium acetate for 1 min, and a colorant was absorbed with filter paper. After drying (overnight at 60° C.), the sample was placed under the transmission electron microscope for observation. It can be clearly seen from FIG. 3 that the Gem-PPG60 micelle (A in FIG. 3) had a smaller particle size than Gem-PPG100 (B in FIG. 3) and Gem-PP100 (C in FIG. 3), exhibited good nano-micelle morphologies, and presented relatively uniform spherical structures.

### 3.Particle Size and Surface Potential Test for Micelles

The particle size distribution and surface potentials of the gemcitabine-loaded micelle samples prepared in Examples 1-2 and Comparative example 1 were measured at 25° C. using a BI-200 SM dynamic light scattering system (Brookhaven Instruments). Scattered light was detected at 90° and collected on an autoaccelerator. For each set of samples, an average of three measurements was taken, as shown in Table 1. A smaller PDI value indicated a more uniform micelle size. The surface of the micelle had electronegativity due to the existence of a carboxyl group. For the Gem-PPG60 and Gem-PPG100 micelles, due to the modification of GCA, the carboxyl groups decreased, the electronegativity was reduced, and the lower electronegativity was more conducive to the adsorption of the micelles on the surface of a small intestinal cell membrane, which promoted the absorption of the micelles by the small intestine, thereby improving the oral bioavailability of the micelles.

TABLE 1
Particle sizes and surface potentials
of three types of gemcitabine micelles

| Samples | Particle size (nm) | PDI | Zeta-potential (mV) |
|---|---|---|---|
| Gem-PPG60 | 66.32 ± 4.16 | 0.062 | −23.2 ± 0.7 |
| Gem-PPG100 | 120.79 ± 9.17 | 0.086 | −21.3 ± 0.5 |
| Gem-PP100 | 114.42 ± 6.31 | 0.11 | −48.7 ± 2.1 |

As can be seen from the above table, Gem-PPG60 had the smallest particle size, relatively uniform micelle size and lowest surface potential electronegativity, and was more conducive to intestinal absorption and higher oral bioavailability.

### 4. Determination for Encapsulation Rates and Drug Loading Capacities of Micelles

High performance liquid chromatography (HPLC) was used to determine the drug content of gemcitabine in the micelles prepared in Examples 1-2 and Comparative example 1. 0.2 mL of micelle sample was taken with a pipette and lyophilized on a lyophilizer to obtain solid mass. The weighed lyophilized micelle solid was redissolved. Detection was performed at a wavelength of 268 nm by using a Phenomenex chromatography column, using 5-bromouracil as an internal standard, using an acetonitrile-0.1% trifluoroacetic acid solution with a ratio of 3:97 as a mobile phase, and setting a flow rate as 1.0 mL/min.

The detection results were shown in Table 2.

TABLE 2
Encapsulation rates and drug loading capacities
of three types of gemcitabine micelles

| Samples | Encapsulation rate (%) | Drug loading capacity (%) |
|---|---|---|
| Gem-PPG60 | 71.2 | 11.7 |
| Gem-PPG100 | 72.1 | 11.2 |
| Gem-PP100 | 64.9 | 9.6 |

As can be seen from the above table, the encapsulation rates and drug loading capacities of the Gem-PPG60 and Gem-PPG100 micelles were both higher than that of a Gem-PP100 micelle unmodified with GCA. 5. In vitro release test for micelles

In vitro release effects of three types of gemcitabine micelles Gem-PPG60, Gem-PPG100 and Gem-PP100 prepared in Examples 1-2 and Comparative example 1 were determined by a dialysis method. Each micelle was dispersed in distilled water and a suspension was placed in a dialysis membrane bag (molecular weight cutoff value of 14 kDa). The bag was sealed and then immersed in PBS (20 mL, 2% Tween 80, pH 7.4). Gemcitabine was released from the micelle using an air bath shaker at 37° C. An external solution was sampled at a predetermined interval (0-120 h), 3 mL each time, and the sampled external solution was replaced with fresh buffer. The concentration of gemcitabine in each sample (n=3) was determined by HPLC and the results were shown in FIG. 4. Within 120 hours, the release rates of Gem-PPG60, Gem-PPG100 and Gem-PP100 were 42.7±2.1%, 57.2±3.1% and 62.5±3.2%, respectively. This in vitro experiment indicated that the Gem-PPG60 micelle modified by GCA and having a particle size of 60 nm was released more slowly, and thus had a better sustained-release effect on gemcitabine.

### III. Pharmacokinetic Experiment for Micelles in Rats

Male Sprague-Dawley (SD) rats weighed 200±20 g were purchased from Guangdong Medical Experimental Animal Center (Guangdong, China), with a feeding temperature of 25+1° C. and a humidity of 50±10%, allowing free access to food and water. Twelve animals were divided into four groups (n=3) on average. The first group was injected intravenously with the Gem-PPG100 micelle (prepared in Example 2), and the second and third groups were administrated intragastrically with the Gem-PPG100 micelle (prepared in Example 2) and the Gem-PPG60 micelle (prepared in Example 1). The last group was administered orally with the unmodified Gem-PP100 micelle (prepared in Comparative example 1). All groups accepted gemcitabine with a dose of 10 mg/kg. Blood samples were collected at a predetermined interval, and 0.2 mL per sample was used for anticoagulation of sodium heparin and stored at −80° C. until analysis. Gemcitabine in plasma was extracted after addition of 1 mL of organic solvent (methanol:acetonitrile=1:9), vortexed for 2 min and centrifuged at 6000 r/min for 5 min. The supernatant was lyophilized, redissolved with 200 μL of ammonium acetate buffer (pH 5.5), vortexed for 2 min and centrifuged at 6000 r/min for 5 min, and 50 μL of supernatant was collected and determined by HPLC. Pharmacokinetic parameters were determined using Origin8.5 (OriginLab, USA). Pharmacokinetic characterization data of the three types of gemcitabine micelles were shown in Table 3. Parameters such as maximum plasma concentration (Cₘₐₓ), time to reach Cₘₐₓ (Tₘₐₓ), half-life (T_{1/2}), total area under a curve (AUC), bioavailability (F %) were calculated directly from a pharmacokinetic map.

TABLE 3
Pharmacokinetic characterization data of three types of gemcitabine micelles

| | Gem-PPG100 | Gem-PPG100 | Gem-PPG60 | Gem-PP100 |
|---|---|---|---|---|
| PK parameters | (intravenous injection) | (oral) | (oral) | (oral) |
| Cₘₐₓ (μg/mL) | 142 ± 6.7 | 21 ± 0.0 | 22 ± 2.3 | 5 ± 0.5 |
| Tₘₐₓ (h) | 0 | 4.0 ± 0 | 4.0 ± 0 | 4.0 ± 0 |
| AUC_{0-inf} | 166 ± 21 | 114 ± 21 | 134 ± 12 | 32 ± 3 |
| (μg · h/mL) | | | | |
| T_{1/2} (h) | 0.53 ± 0.06 | 6.97 ± 0.3 | 6.13 ± 0.1 | 6.33 ± 0.3 |
| Bioavailability | 100 | 68.7 | 80.7 | 19.3 |
| (F %) | | | | |

As can be seen from the above table, the oral bioavailability (F %) of the Gem-PP100 micelle unmodified with GCA was 19.3%, while the oral bioavailability of the Gem-PPG60 and Gem-PPG100 micelles modified by GCA was 80.7% and 68.7%, respectively, demonstrating that oral administration of the Gem-PPG micelle greatly improved the bioavailability of gemcitabine, and the effect of Gem-PPG60 was better than that of Gem-PPG100.

### IV. In Vitro Cytotoxicity Test for Micelles

Human pancreatic cancer cell lines BxPC-3 and Mia-paca-2 were both purchased from the American Type Culture Collection (ATCC, Rockville, MD). According to official guidelines, all cell lines were routinely subcultured in a 37° C., 5% CO₂ air environment, and cells grown during an exponential growth phase were collected and counted for later use. The anti-tumor efficacy was analyzed by a CTG method. Cells were stained with trypan blue and then counted with a hemocytometer. The cells were adjusted to an appropriate cell density, 135 μL of cell suspension was then poured into an analysis plate, and 135 μL of analysis medium was added to blank wells. The culture plate was incubated overnight in 5% CO₂ and 95% air at 37° C. and 100% relative humidity. A test sample was diluted (10 times a working concentration), 15 μL of diluted solution was then added to microwells, and the analysis plate was put back in an incubator, and incubated for 5 days. To detect cell viability values, 75 μL of CellTiter Glo reagent (Promega, USA) was added to each well on Day 1 and Day 5, the plate was gently shaken for 10 min at room temperature, and then the luminescence was recorded on a 2104 EnVision microplate reader (EnVision, USA). The results were shown in Table 4.

TABLE 4
In vitro cytotoxicity of Gem-PPG60 micelle (prepared in Example
1), Gem-PP100 micelle (prepared in Comparative example 1), and
commercial gemcitabine injection on BxPC-3 and Mia Paca-2

| Cell lines | Samples | Relative GI₅₀(nM) |
|---|---|---|
| BxPC-3 | Gem-PP100 | 5.8 |
| | Gem-PPG60 | 5.3 |
| | Gemcitabine injection | 4.0 |
| Mia Paca-2 | Gem-PP100 | 11.9 |
| | Gem-PPG60 | 11.0 |
| | Gemcitabine injection | 7.3 |

In this table, GI₅₀ represented a half maximal inhibitory concentration value.

As can be seen from the above table, Gem-PPG60, Gem-PP100 and gemcitabine injections all exhibited certain dose-dependent toxicity within 120 h: GI₅₀ was 5.3 nM, 5.8 nM, and 4.0 nM in a BxPC-3 cell line, and GI₅₀ was 11.0 nM, 11.9 nM, and 7.3 nM in a Mia-Paca-2 cell line, respectively. The GI₅₀ value of the Gem-PPG60 micelle modified by GCA was lower than that of the Gem-PP100 micelle unmodified by GCA, indicating the effectiveness of GCA modification on the micelles. In addition, because gemcitabine was encapsulated in the micelles, the GI₅₀ value of the Gem-PPG60 micelle was slightly higher than that of the commercial gemcitabine solution, but there was no significant difference, proving that the Gem-PPG60 micelle also had good cytotoxic activity in vitro compared with the commercial gemcitabine solution.

### V. Pharmacokinetic Experiment for Micelles in Mice

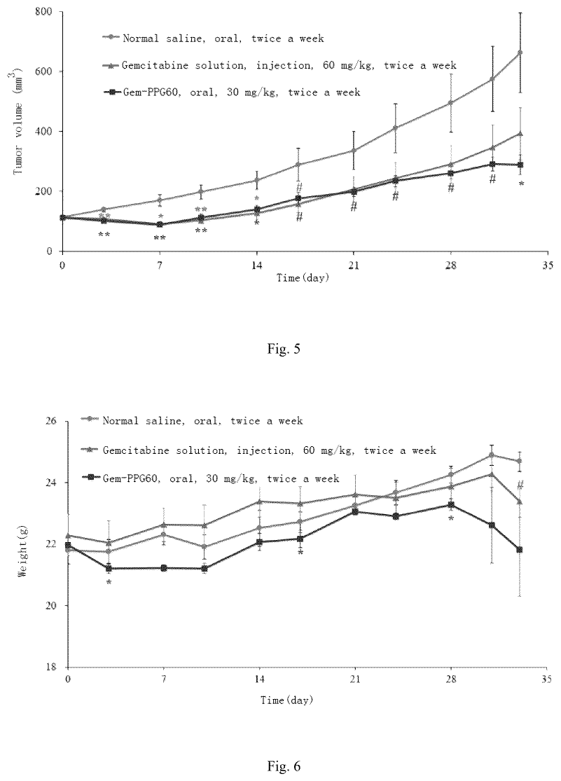

200 μL of RPMI 1640 medium (Gibco, USA) containing BxPC-3 tumor cells was injected subcutaneously at a concentration of 1×10⁷ cells/0.2 mL into the dorsal right neck of the mouse and maintained until a solid tumor was formed. To evaluate a therapeutic effect, Balb/c nude mice were randomly divided into three groups (5 in each group). The mice were administrated with normal saline (Saline, negative control group) 1 h before eating, injected intraperitoneally with a commercial gemcitabine hydrochloride injection (positive control group, 60 mg/kg, BIW), and administrated orally with the Gem-PPG60 micelle (experimental group, 30 mg/kg, BIW), respectively, and tumor sizes (as shown in FIG. 5) and weights were measured twice a week (as shown in FIG. 6). The experiment was terminated on Day 33, the mice were euthanized and the tumor volume was obtained. As shown in FIG. 5, the volume progression of tumors in the oral Gem-PPG60 micelle group was slowest. Tumor growth inhibition rates (TGI) were 49.1% and 68.1% in the gemcitabine solution group and the Gem-PPG60 micelle group, respectively, and calculated according to a formula TGI (%)=[1−(T₃₃−T₀)/(V₃₃−V₀)]×100%, wherein T₃₃ represented an average tumor volume of the experimental group on Day 33, T₀ represented an average tumor volume before the start of the experimental group, V₃₃ represented an average tumor volume of the control group at Day 33, and V₀ represented an average tumor volume before the start of the control group. It was indicated that the oral administration of the Gem-PPG60 micelle inhibited tumor growth more effectively than injection administration of commercial gemcitabine hydrochloride in a BxPC-3 mouse model, even at half the dose (30 mg/kg). In addition, FIG. 6 showed that there was no significant difference in weights of mice in the three groups, indicating that the oral administration of the Gem-PPG60 micelle had certain safety.

In summary, according to the present invention, the PLGA-PEG high polymer modified by GCA was adopted to wrap the hydrophobic small molecule drug (e.g., gemcitabine) by an ultrasonic emulsification method, thereby obtaining the drug-loaded micelle composition. The drug-loaded micelle self-emulsified and wrapped hydrophobic small molecules by using amphiphilic properties of the PPG high polymer (hydrophobic for PLGA, hydrophilic for GCA), with an encapsulation rate of more than 70% and a drug loading rate of more than 11%, achieving good sustained-release effect and greatly improved oral bioavailability. In addition, through in vitro cytotoxicity tests and in vivo pharmacodynamic experiments for mice, it was proved that the oral administration of a chemotherapy drug gemcitabine can be realized and a therapeutic effect on pancreatic cancers can be exerted, while an administration dose was reduced.

Although the contents of the present invention have been described in detail by the preferred examples above, it should be recognized that the above description should not be considered as a limitation on the present invention. After a person skilled in the art has read the above contents, a variety of modifications and substitutions made for the present invention will be obvious. Therefore, the protection scope of the present invention should be defined by the appended claims.

1. An oral drug-loaded micelle composition, comprising:
a PLGA-PEG high polymer modified by glycocholic acid (GCA), marked as PLGA-PEG-GCA; and
a hydrophobic small molecule drug, wherein
a hydrophobic end PLGA of GCA-PLGA-PEG is aggregated to wrap the hydrophobic small molecule drug, and a hydrophilic end of the GCA-PLGA-PEG extends toward a direction away from the hydrophobic end PLGA to form a spherical drug-loaded micelle.

2. The oral drug-loaded micelle composition according to claim 1, wherein in PLGA-PEG-GCA, a PLGA fragment has a molecular weight of 8000 Da-12000 Da; and a PEG fragment has a molecular weight of 3000 Da-7000 Da.

3. The oral drug-loaded micelle composition according to claim 1, wherein in PLGA-PEG-GCA, the spherical drug-loaded micelle has a particle size of 20-200 nm.

4. The oral drug-loaded micelle composition according to claim 1, wherein the hydrophobic small molecule drug comprises one or more of the followings: gemcitabine, doxorubicin, daunorubicin, epirubicin, azathramycin, valrubicin, anthracyclines, actinomycin-D, bleomycin, mitomycin-C, cyclophosphamide, mecloxamine, uramustine, melphalan, chloramphenicol, ifosfamide, bendamustine, carmustine, lomustine, streptomycin, busulfan, dacarbazine, temozolomide, thio-TEPA, altretamine, cisplatin, carboplatin, nedaplatin, oxaliplatin, satraplatin, triplatinum tetranitrate, 5-fluorouracil, 6-mercaptopurine, capecitabine, cladribine, clofarabine, cytarabine, fluorouracil, fludarabine, hydroxyurea, methotrexate, pemetrexed, pentostatin, thioguanine; camptothecin, topotecan, irinotecan, etoposide, teniposide, mitoxantrone, paclitaxel, docetaxel, ixabepilone, vinblastine, vincristine, vindesine, vinorelbine, estradiol and derivatives thereof.

5. The oral drug-loaded micelle composition according to claim 1, wherein the hydrophobic small molecule drug in the oral drug-loaded micelle has a drug loading rate of 11%-20% by mass.

6. The oral drug-loaded micelle composition according to claim 1, wherein the hydrophobic small molecule drug in the oral drug-loaded micelle has an encapsulation rate of 70%-90% by mass.

7. A preparation method of the oral drug-loaded micelle composition according to claim 1, comprising:
step 1, preparing PLGA-PEG-GCA from PLGA-PEG-COOH modified by glycocholic acid (GCA); and
step 2, adding a hydrophobic small molecule drug and PLGA-PEG-GCA to an organic solvent in a mass ratio of 1:5 to 1:10, which are dissolved, then dropwise added to distilled water under the action of ultrasounds, emulsified to form a micelle for wrapping the hydrophobic small molecule drug, purified and filtered to obtain the oral drug-loaded micelle.

8. The preparation method of the oral drug-loaded micelle composition according to claim 7, wherein the step 1 comprises:
step 1.1, with glycocholic acid (GCA) and ethylenediamine (EDA) as raw materials, reacting at room temperature under the action of a condensing agent to prepare GCA-EDA; and
step 1.2, activating a carboxyl group of PLGA-PEG-COOH in the atmosphere of nitrogen, adding GCA-EDA, reacting at room temperature for 6 h-12 h, and purifying to obtain the PLGA-PEG-GCA.

9. The preparation method of the oral drug-loaded micelle composition according to claim 7, wherein the purifying step comprises: performing dialysis by distilled water at a molecular weight of 12 k Da-16 k Da and purifying for 24 h-72 h to remove the hydrophobic small molecule drug free outside the micelle.

10. The preparation method of the oral drug-loaded micelle composition according to claim 8, wherein PLGA is also added in the step 2.
